(19) United States
(12) Patent Application Publication
(10) Pub. No.: US 2023/0330403 A1
(43) Pub. Date: Oct. 19, 2023

(54) MANUFACTURING METHOD OF MICRONEEDLE BIOSENSOR

(71) Applicant: PEKING UNIVERSITY, Beijing (CN)

(72) Inventor: Yue Cui, Beijing (CN)

(21) Appl. No.: 18/134,730

(22) Filed: Apr. 14, 2023

(30) Foreign Application Priority Data
Apr. 15, 2022 (CN) 202210393852.6

(51) Int. Cl. A61M 37/00

(52) U.S. Cl. CPC A61M 37/0015; A61M 2037/0046; A61M 2037/0053

(57) ABSTRACT

An embodiment of the present application provides a manufacturing method of a microneedle biosensor, and relates to the technical field of medical instruments. The method includes: providing a mold, where a microneedle array is formed on the mold; casting a liquid polymer material on the mold, and drying and then demolding to form a sensor substrate, where a hollow microneedle array is provided on the sensor substrate; penetrating through a tip of each microneedle in the hollow microneedle array; and forming a working electrode and a counter electrode on the sensor substrate, where the working electrode and the counter electrode each cover a part of the hollow microneedle array.

### CROSS REFERENCE TO RELATED APPLICATION

This patent application claims the benefit and priority of Chinese Patent Application No. 202210393852.6, filed with the China National Intellectual Property Administration on Apr. 15, 2022, the disclosure of which is incorporated by reference herein in its entirety as part of the present application.

### TECHNICAL FIELD

Embodiments of the present application relate to the technical field of medical instruments, and in particular, to a manufacturing method of a microneedle biosensor.

### BACKGROUND

A microneedle array biosensor is implemented by depositing a layer of biological recognition molecules on the surface of a microneedle array. When the microneedle array biosensor penetrates into a human body, the biological recognition molecules on the microneedle array react with biological molecules in interstitial fluid to generate electrons, so as to express a target concentration as an electrical signal. The microneedle array biosensor does not come into contact with a subcutaneous pain nerve when detecting the human body, and has the advantages of being painless, simple to operate and safe, causing no infection, etc.

However, a current microneedle sensor is not ideal in biodegradability or biocompatibility.

### SUMMARY

An embodiment of the present application provides a manufacturing method of a microneedle biosensor, aiming to improving the biodegradability or biocompatibility of a microneedle sensor.

According to a first aspect of embodiments of the present application, a manufacturing method of a microneedle biosensor is provided, the method including:
providing a mold, where a microneedle array pattern or a microneedle hole array pattern is formed on the mold;
casting a liquid polymer material on the mold, and drying and then demolding to form a sensor substrate, where a hollow microneedle array is provided on the sensor substrate, and the liquid polymer material is a biodegradable material or a biocompatible material;
penetrating through a tip of each microneedle in the hollow microneedle array; and
forming a working electrode and a counter electrode on the sensor substrate, where the working electrode and the counter electrode each cover a part of the hollow microneedle array.

Optionally, the liquid polymer material is selected from the group consisting of chitosan, polylactic acid, silk fibroin and thermoplastic polyurethane.

Optionally, in a process of forming the working electrode and the counter electrode on the sensor substrate, the method includes:
forming the working electrode and the counter electrode on a protruding side of the hollow microneedle array on the sensor substrate; or
forming the working electrode and the counter electrode on a recessed side of the hollow microneedle array on the sensor substrate.

Optionally, the working electrode includes an electrode layer, a Prussian blue layer and an enzyme layer that are stacked on the sensor substrate; and in a process of forming the working electrode on the sensor substrate, the method includes:
forming the electrode layer on the sensor substrate by means of a vapor deposition or sputtering process, so that the electrode layer covers a part of the hollow microneedle array;
forming the Prussian blue layer on a side of the electrode layer far away from the sensor substrate;
forming the enzyme layer on a side of the Prussian blue layer far away from the electrode layer; and
forming a biocompatible polymer layer on a side of the enzyme layer far away from the Prussian blue layer.

Optionally, a partition area is provided between the working electrode and the counter electrode. Before forming the working electrode and the counter electrode on the sensor substrate, the method further includes:

forming a water-proof layer on the sensor substrate.

Optionally, in a process of penetrating through the tip of each microneedle in the hollow microneedle array, the method includes:

penetrating through the tip of each microneedle in the hollow microneedle array by means of a metal needle array, where steel needles of the metal needle array are in a one-to-one correspondence with microneedles of the hollow microneedle array.

Optionally, the mold is made of polydimethylsiloxane.

According to a second aspect of embodiments of the present application, use of a microneedle biosensor is provided, including a microneedle sensor manufactured by means of the manufacturing method of a microneedle biosensor according to the first aspect, the microneedle sensor including a sensor substrate and a hollow microneedle array formed on the sensor substrate, where

the hollow microneedle array is used as an injection channel for drug injection.

Beneficial effects:

The present application provides a manufacturing method of a microneedle biosensor, including: casting a liquid polymer material on a mold with a microneedle array pattern or a microneedle hole array pattern, drying and then demolding so as to form a sensor substrate with a hollow microneedle array, then penetrating through a tip of each microneedle in the hollow microneedle array, and forming a working electrode and a counter electrode on the sensor substrate, so that the manufacture of a microneedle sensor is completed; by forming the sensor substrate with the hollow microneedle array directly by means of the mold, the sensor substrate may be made of a biodegradable material or a biocompatible material, so that the biodegradability or biocompatibility of the microneedle sensor can be effectively improved.

### BRIEF DESCRIPTION OF THE DRAWINGS

To describe the technical solutions in the embodiments of the present application more clearly, the following briefly describes the accompanying drawings required for describing the embodiments of the present application. Apparently, the accompanying drawings in the following description show merely some embodiments of the present application, and those of ordinary skill in the art may still derive other accompanying drawings from these accompanying drawings without creative efforts.

FIG. 1 is a flowchart of steps of a manufacturing method of a microneedle biosensor according to an embodiment of the present application;

FIG. 2 is a flowchart of steps of manufacturing a mold in a manufacturing method of a microneedle biosensor according to an embodiment of the present application;

FIG. 3 is a flowchart of steps of manufacturing a sensor substrate in a manufacturing method of a microneedle biosensor according to an embodiment of the present application;

FIG. 4 is a flowchart of steps of manufacturing a working electrode in a manufacturing method of a microneedle biosensor according to an embodiment of the present application;

FIG. 5 is a scanning electron microscope (SEM) image of one side of a microneedle biosensor according to an embodiment of the present application;

FIG. 6 is an SEM image of the other side of a microneedle biosensor according to an embodiment of the present application;

FIG. 7 is a schematic structural diagram of a microneedle biosensor according to an embodiment of the present application;

FIG. 8 is a schematic structural diagram of a cross-section taken along line a-a′ in FIG. 7;

FIG. 9 is a schematic structural diagram of another microneedle biosensor according to an embodiment of the present application;

FIG. 10 is a schematic structural diagram of a cross-section taken along line b-b′ in FIG. 9;

FIG. 11 is a schematic structural diagram of another microneedle biosensor according to an embodiment of the present application;

FIG. 12 is a schematic structural diagram of another microneedle biosensor according to an embodiment of the present application;

FIG. 13 is a time-varying curve graph of a current generated by a microneedle biosensor according to an embodiment of the present application when detecting glucose concentrations in simulated interstitial fluids having different concentrations; and

FIG. 14 is a calibration curve graph showing a change in current amplitude generated by a microneedle biosensor according to an embodiment of the present application with a glucose concentration when detecting glucose concentrations in simulated interstitial fluids having different concentrations.

Description of reference numerals: 1. Sensor substrate; 2. Hollow microneedle array; 3. Working electrode; 4. Counter electrode; 5. Reference electrode; A. Partition area.

### DETAILED DESCRIPTION OF THE EMBODIMENTS

The technical solutions in the embodiments of the present application are clearly and completely described below with reference to the accompanying drawings in the embodiments of the present application. Apparently, the described embodiments are merely some rather than all of the embodiments of the present application. All other embodiments obtained by those of ordinary skill in the art based on the embodiments of the present application without creative efforts should fall within the protection scope of the present application.

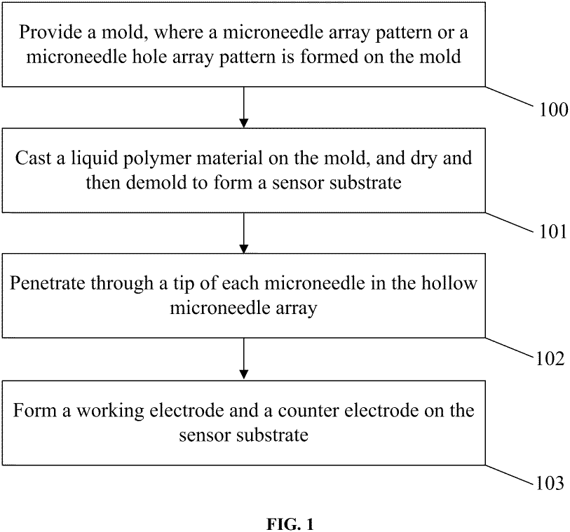

FIG. 1 is a flowchart of steps of a manufacturing method of a microneedle biosensor. As shown in FIG. 1, the present application provides a manufacturing method of a microneedle biosensor, including the following steps.

Step 100: Provide a mold, where a microneedle array pattern or a microneedle hole array pattern is formed on the mold.

Specifically, before the mold with the microneedle array pattern or the microneedle hole array pattern is manufactured, a molding template with a negative pattern needs to be provided first, where the negative pattern refers to a pattern set for forming the microneedle array pattern or the microneedle hole array pattern. The molding template with the negative pattern may be a purchased existing template or be made by using a laser cutting machine.

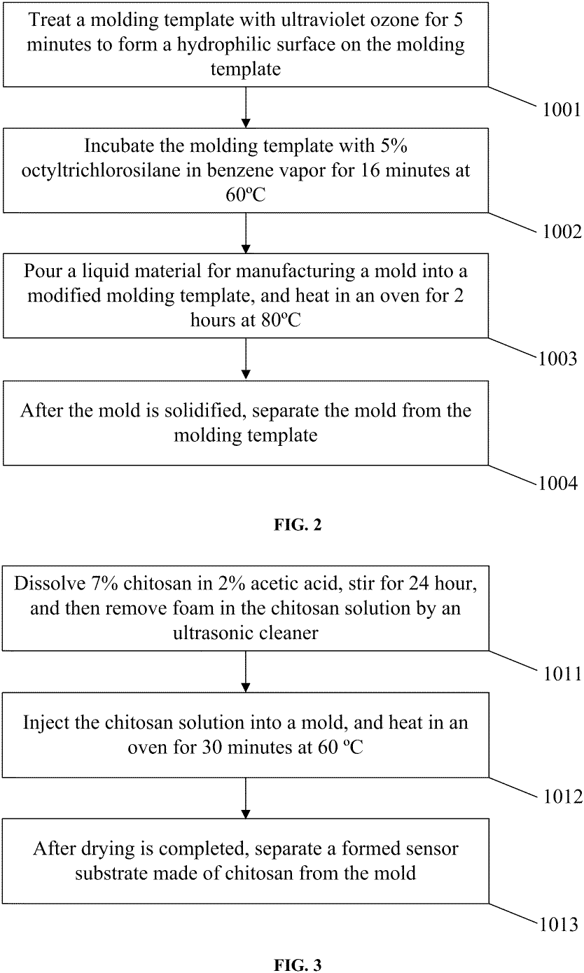

FIG. 2 shows a method for manufacturing a mold with a microneedle pattern array according to an embodiment of the present application, the method including the following steps.

Step 1001: Treat a molding template with ultraviolet ozone for 5 minutes to form a hydrophilic surface on the molding template.

Step 1002: Incubate the molding template with 5% octyltrichlorosilane in benzene vapor for 16 minutes at 60° C. to silanize the surface of the molding template.

Step 1003: Pour a liquid material for manufacturing a mold into a modified molding template, and heat in an oven for 2 hours at 80° C.

Step 1004: After the mold is solidified, separate the mold from the molding template to obtain a mold with a microneedle array.

In the specific application, the mold and the molding template may be both made of polydimethylsiloxane, which can reduce required costs of the mold and the molding template, thereby further reducing the manufacturing cost of the microneedle biosensor. In addition, in the above steps, the heating temperature and heating time for manufacturing the mold can be adjusted according to the material of the mold or the molding template.

Step 102: Cast a liquid polymer material on the mold, and dry and then demold to form a sensor substrate 1, where a hollow microneedle array 2 is provided on the sensor substrate 1, and the liquid polymer material is a biodegradable material or a biocompatible material.

Specifically, the liquid polymer materials may be a biodegradable material, such as chitosan, polylactic acid and silk fibroin, or may be a biocompatible material, such as thermoplastic polyurethane. When a biodegradable material is used, the microneedle sensor has degradability and can be naturally decomposed after use. When a biocompatible material is used, the microneedle sensor has high biocompatibility, thereby avoiding damage to a human body during use.

FIG. 3 shows a method for manufacturing a sensor substrate made of chitosan, the method including the following steps.

Step 1011: Dissolve 7% chitosan in 2% acetic acid, stir for 24 hours by using an electromagnetic stirrer, and then remove foam in the chitosan solution by means of ultrasonic wave.

Step 1012: Inject the chitosan solution into a mold, and heat in an oven for 30 minutes at 60° C.

Step 1013: After drying is completed, separate a formed sensor substrate 1 made of chitosan from the mold.

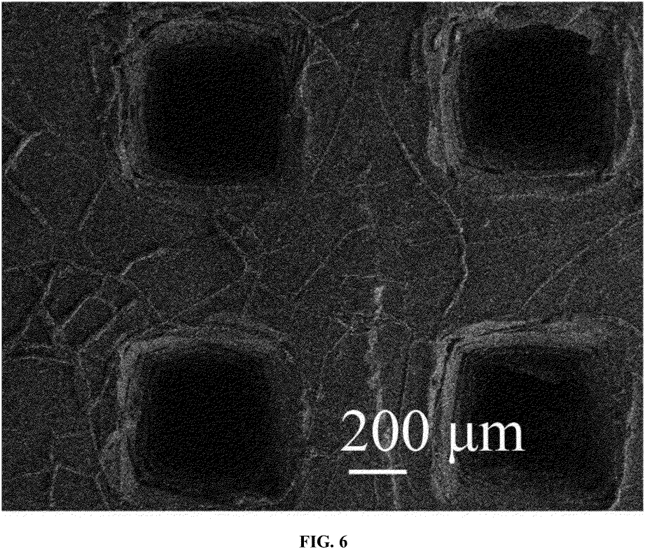

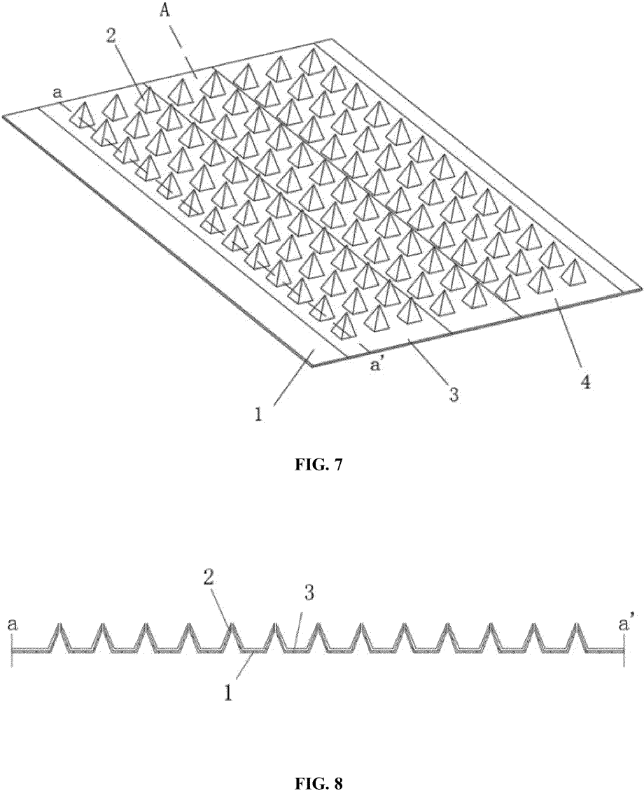

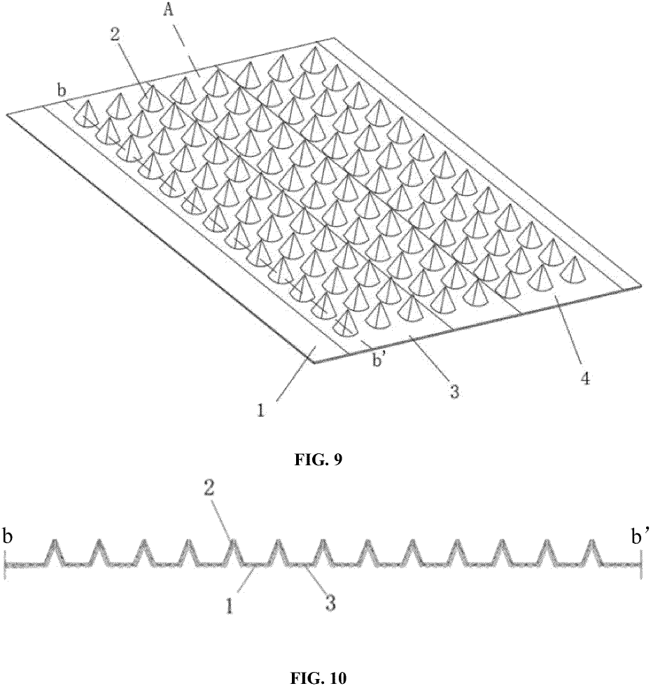

In this way, as shown in FIGS. 5 and 6, a sensor substrate 1 with a hollow microneedle array 2 is formed. As shown in FIGS. 7 and 9, each microneedle of the hollow microneedle array 2 may be in the shape of a pyramid or a cone.

In addition, when the sensor substrate 1 is made of another material, the heating time and the heating temperature for the sensor substrate can also be adjusted based on the material.

Step 102: Penetrate through a tip of each microneedle in the hollow microneedle array 2.

Specifically, a metal needle array may be selected for penetration, and the metal needle array needs to be aligned with the sensor substrate 1 in size and position, that is, metal needles in the metal needle array are in a one-to-one correspondence with microneedles in the hollow microneedle array 2.

In specific application, a stainless steel needle array may be selected to penetrate through tips of each microneedle array in the hollow microneedle array 2, so that an obvious hole is formed in the tip of each microneedle, and thus when the microneedle biosensor is used for detection, effective substances in the tested solution can flow into the hollow microneedles through the holes in the tips of the microneedles. In another embodiment, a needle array made of another hard material may also be selected to penetrate through the hollow microneedle array 2.

Step 103: Form a working electrode 3 and a counter electrode 4 on the sensor substrate 1, where the working electrode 3 and the counter electrode 4 each cover a part of the hollow microneedle array 2.

Specifically, as shown in FIG. 8, the working electrode 3 and the counter electrode 4 may be manufactured on a protruding side of the hollow microneedle array 2 on the sensor substrate 1; and as shown in FIG. 10, the working electrode 3 and the counter electrode 4 may also be manufactured on a recessed side of the hollow microneedle array 2 on the sensor substrate 1. In addition, when the working electrode 3 and the counter electrode 4 are manufactured on the protruding side of the hollow microneedle array 2, it is optional not to penetrate through the hollow microneedle array 2, because in this case, the microneedles only need to be in contact with the tested solution, and the tested solution does not need to flow into the hollow microneedle array 2.

In addition, the working electrode 3 includes an electrode layer, a Prussian blue layer, an enzyme layer and a biocompatible polymer layer that are stacked on the sensor substrate 1, where the electrode layer may be made of Au. The counter electrode 4 usually includes an electrode layer.

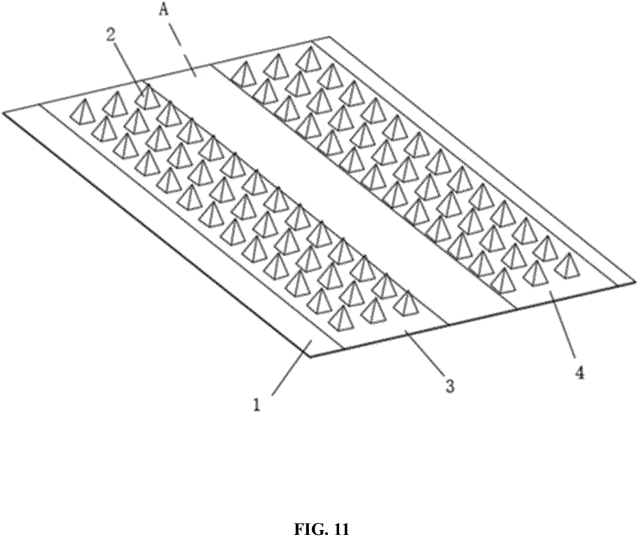

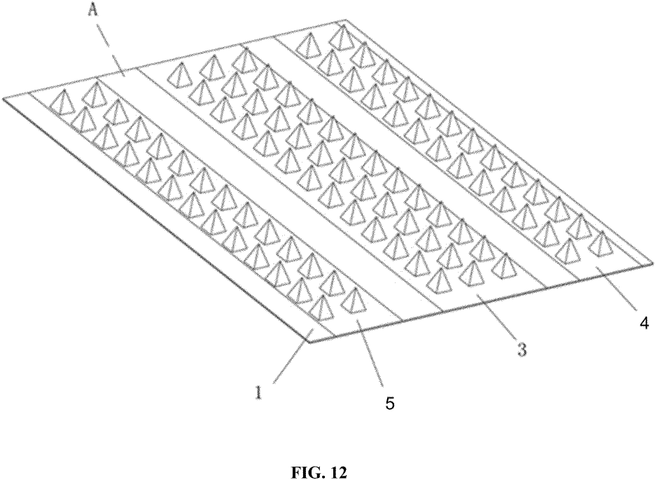

In specific application, as shown in FIG. 11, the counter electrode 4 may include only one counter electrode 4. In this case, the counter electrode 4 may have functions of connecting a circuit and stabilizing a voltage. The counter electrode 4 may be made of Ag/AgCl. As shown in FIG. 12, the electrode 2 may also include a reference electrode 5 and a counter electrode 4. In this case, the reference electrode 5 has a function of stabilizing a voltage, and the counter electrode 4 has a function of connecting a circuit. The counter electrode 4 may be made of Au or Pt. The reference electrode 5 may be made of Ag/AgCl.

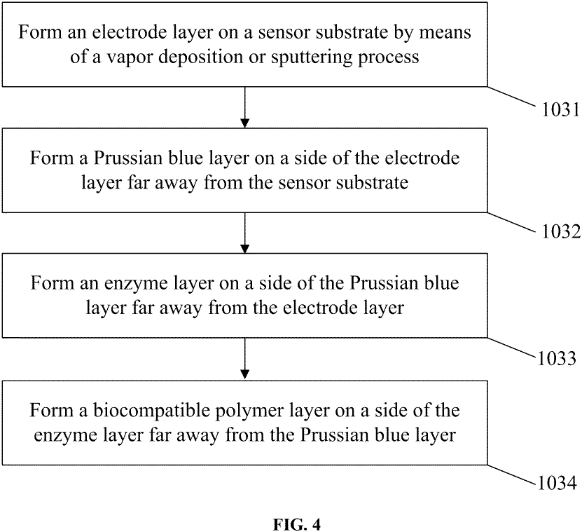

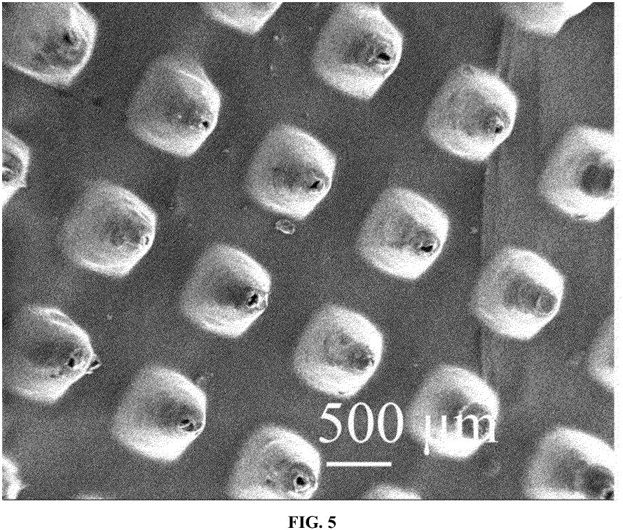

FIG. 4 shows a method for manufacturing a working electrode according to an embodiment of the present application, the method including the following steps.

Step 1031: Form an electrode layer on a sensor substrate 1 by means of a vapor deposition or sputtering process, so that the electrode layer covers a part of a hollow microneedle array 2.

Specifically, when the electrode layer is formed by means of the vapor deposition or sputtering process, a mask plate with holes needs to be selected for vapor deposition or sputtering, so as to implement the patterning of the electrode layer.

Step 1032: Form a Prussian blue layer on a side of the electrode layer far away from the sensor substrate 1.

Specifically, the Prussian blue layer is formed on the sensor substrate 1 by means of an electroplating process.

Step 1033: Form an enzyme layer on a side of the Prussian blue layer far away from the electrode layer.

Specifically, the enzyme layer is formed by covering the Prussian blue layer with liquid enzyme solution, and then heating and drying the liquid enzyme.

Step 1034: Form a biocompatible polymer layer on a side of the enzyme layer far away from the Prussian blue layer.

Specifically, the biocompatible polymer layer is formed by covering the enzyme layer with a liquid biocompatible polymer, and then heating and drying the liquid biocompatible polymer. The biocompatible polymer layer may be made of perfluorosulfonic acid, and the biocompatible polymer layer can prevent the Prussian blue layer from damaging the human body.

In this way, when the working electrode 3 comes into contact with the tested solution, the enzyme can react with corresponding analyte in the tested solution, and a product is generated from the enzymatic reaction. The product undergoes oxidation or reduction reaction on the working electrode 3 to generate a change in the electrical signal.

The finally formed microneedle biosensor is shown in FIGS. 7 and 9. The manufacture of the microneedle sensor is completed by casting a liquid polymer material on a mold with a microneedle array pattern or a microneedle hole array pattern, drying and then demolding so as to form a sensor substrate 1 with a hollow microneedle array 2, then penetrating through a tip of each microneedle in the hollow microneedle array 2, and forming a working electrode 3 and a counter electrode 4 on the sensor substrate 1; by forming the sensor substrate 1 with the hollow microneedle array 2 directly by means of the mold, the sensor substrate may be made of a biodegradable material or a biocompatible material, so that the biodegradability or biocompatibility of the microneedle sensor can be effectively improved.

In addition, as shown in FIGS. 7 and 9, in order to prevent short circuit caused by an excessively small distance between the working electrode 3 and the counter electrode 4, a partition area A is provided on the sensor substrate 1 and between the working electrode 3 and the counter electrode 4, that is, the hollow microneedle array 2 located in the partition area A is not covered with the working electrode 3 or the counter electrode 4. In addition, as shown in FIG. 11, the hollow microneedle array 2 may not be formed in the partition area A.

In an embodiment, when the sensor substrate 1 is made of chitosan, in order to prevent the damage to the hollow microneedle array 2 caused by the proneness of the chitosan to water absorption and expansion, a water-proof layer may be further formed on the sensor substrate 1 before the working electrode 3 and the counter electrode 4 are formed, so as to prevent the sensor substrate 1 from coming into contact with the solution.

In specific application, the water-proof layer may be made of parylene.

In addition, when the sensor substrate 1 is made of a water-insoluble material, the water-proof layer may not be provided.

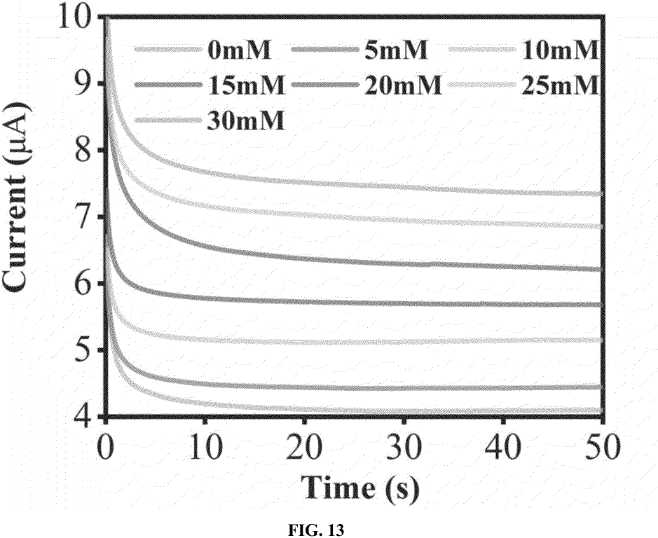

FIG. 13 is a time-varying curve graph of a current generated by a microneedle biosensor according to an embodiment of the present application when detecting glucose concentrations in simulated interstitial fluids having different concentrations. It can be seen from FIG. 13 that a higher glucose concentration in a simulated tissue fluid indicates a higher current generated by the microneedle biosensor.

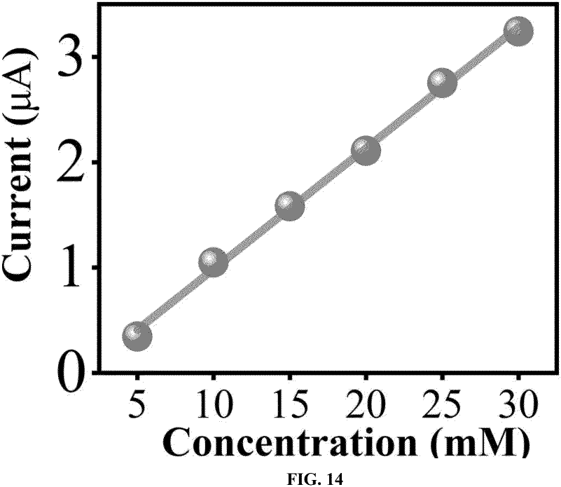

Based on current values at different concentrations obtained in FIG. 13, a coordinate system shown in FIG. 12 can be obtained by taking a glucose concentration in a simulated interstitial fluid as an abscissa axis and a current amplitude increasing with the glucose concentration as an ordinate axis. In addition, current amplitudes at different concentrations are marked in the coordinate system, that is, a plurality of coordinate points in FIG. 14 are marked, and these coordinate points are connected by using a straight line, to finally obtain a calibration curve in FIG. 14.

Finally, based test data, an expression between the current amplitude and the glucose concentration can be obtained as follows: y=0.117x-0.166,

where y represents the current amplitude, and x represents the glucose concentration.

Based on the above expression, when the microneedle sensor according to the embodiment of the present application is used to measure the glucose concentration in an interstitial fluid to be tested, the glucose concentration in the interstitial fluid to be tested can be calculated according to the formula after the measured current value is obtained.

An embodiment of the present application further discloses use of a microneedle biosensor, including a microneedle sensor manufactured by means of the manufacturing method of a microneedle biosensor according to Embodiment 1, the microneedle sensor including a sensor substrate and a hollow microneedle array formed on the sensor substrate, where the hollow microneedle array is used as an injection channel for drug injection.

By forming holes in the microneedles of the hollow microneedle array 2, when the microneedle sensor is used in practice, for example, when the microneedle sensor is used to measure the glucose concentration in the human tissue fluid, the hollow microneedles with the holes in the hollow microneedle array 2 can be used as injection channels to inject a drug into the human body, and a type of an injected drug, a drug injection rate, etc. can be selected based on the glucose concentration tested by the microneedle sensor, thereby making the use of the microneedle sensor more convenient. This is also the main reason why the microneedle sensor needs to form a sensor substrate 1 with a hollow microneedle array 2 in the present application.

It should be noted that, the embodiments in the description are described in a progressive manner. Each embodiment focuses on the difference from other embodiments, and the same and similar parts between the embodiments may refer to each other.

Orientations or position relationships indicated by terms “center”, “upper”, “lower”, “left”, “right”, “vertical”, “horizontal”, “inner”, “outer”, etc. are orientation or position relationships shown in the accompanying drawings, and these terms are only intended to facilitate description of the present application and simplify the description, rather than indicating or implying that the referred apparatus or element must have a specific orientation and be constructed and operated in a specific orientation, and therefore should not be construed as limiting the present application. In addition, relational terms such as “first” and “second” are merely used to distinguish one entity or operation from another entity or operation without necessarily requiring or implying any actual such relationship or order between such entities or operations, and also should not be construed as indicating or implying relative importance. Moreover, the terms “include”, “comprise” or any other variants thereof are intended to cover non-exclusive inclusion, so that a process, method, article or terminal device including a series of elements not only includes those elements, but also includes those elements that are not explicitly listed, or also includes elements inherent to this process, method, article or terminal device. Without more restrictions, the elements defined by the sentence “including a ...” do not exclude the existence of other identical elements in the process, method, article, or terminal device including the elements.

The technical solutions of the present application are described in detail above. Specific examples are used herein to illustrate the principle and implementations of the present application, and the description of the above embodiments is only intended to help understand the present application. The content of the description should not be construed as limiting the present application. In addition, for those of ordinary skill in the art, different forms of changes may be made to the specific implementations and application scope according to the present application, it is not necessary and impossible to exhaust all the implementations herein, and the obvious changes or modifications arising therefrom still fall within the protection scope of the present application.

What is claimed is:

1. A manufacturing method of a microneedle biosensor, comprising:
providing a mold, wherein a microneedle array pattern or a microneedle hole array pattern is formed on the mold;
casting a liquid polymer material on the mold, and drying and then demolding to form a sensor substrate, wherein a hollow microneedle array is provided on the sensor substrate, and the liquid polymer material is a biodegradable material or a biocompatible material;
penetrating through a tip of each microneedle in the hollow microneedle array; and
forming a working electrode and a counter electrode on the sensor substrate, wherein the working electrode and the counter electrode each cover a part of the hollow microneedle array.

2. The manufacturing method of a microneedle biosensor according to claim 1, wherein
the liquid polymer material is selected from the group consisting of chitosan, polylactic acid, silk fibroin and thermoplastic polyurethane.

3. The manufacturing method of a microneedle biosensor according to claim 1, wherein in a process of forming the working electrode and the counter electrode on the sensor substrate, the method comprises:
forming the working electrode and the counter electrode on a protruding side of the hollow microneedle array on the sensor substrate; or
forming the working electrode and the counter electrode on a recessed side of the hollow microneedle array on the sensor substrate.

4. The manufacturing method of a microneedle biosensor according to claim 1, wherein the working electrode comprises an electrode layer, a Prussian blue layer, an enzyme layer and a biocompatible polymer layer that are stacked on the sensor substrate; and in a process of forming the working electrode on the sensor substrate, the method comprises:
forming the electrode layer on the sensor substrate by means of a vapor deposition or sputtering process, so that the electrode layer covers a part of the hollow microneedle array;
forming the Prussian blue layer on a side of the electrode layer far away from the sensor substrate;
forming the enzyme layer on a side of the Prussian blue layer far away from the electrode layer; and
forming the biocompatible polymer layer on a side of the enzyme layer far away from the Prussian blue layer.

5. The manufacturing method of a microneedle biosensor according to claim 1, wherein
a partition area is provided between the working electrode and the counter electrode.

6. The manufacturing method of a microneedle biosensor according to claim 1, wherein before forming the working electrode and the counter electrode on the sensor substrate, the method further comprises:
forming a water-proof layer on the sensor substrate.

7. The manufacturing method of a microneedle biosensor according to claim 1, wherein in a process of penetrating through the tip of each microneedle in the hollow microneedle array, the method comprises:
penetrating through the tip of each microneedle in the hollow microneedle array by a metal needle array, wherein metal needles of the metal needle array are in a one-to-one correspondence with microneedles of the hollow microneedle array.

8. The manufacturing method of a microneedle biosensor according to claim 1, wherein
the mold is made of polydimethylsiloxane.

9. Using method of a microneedle biosensor, comprising a microneedle sensor manufactured by the manufacturing method of a microneedle biosensor according to claim 1, the microneedle sensor comprising a sensor substrate and a hollow microneedle array formed on the sensor substrate, wherein
the hollow microneedle array is used as an injection channel for drug injection.

10. The using method according to claim 9, wherein
the liquid polymer material is selected from the group consisting of chitosan, polylactic acid, silk fibroin and thermoplastic polyurethane.

11. The using method according to claim 9, wherein in a process of forming the working electrode and the counter electrode on the sensor substrate, the method comprises:
forming the working electrode and the counter electrode on a protruding side of the hollow microneedle array on the sensor substrate; or
forming the working electrode and the counter electrode on a recessed side of the hollow microneedle array on the sensor substrate.

12. The using method according to claim 9, wherein the working electrode comprises an electrode layer, a Prussian blue layer, an enzyme layer and a biocompatible polymer layer that are stacked on the sensor substrate; and in a process of forming the working electrode on the sensor substrate, the method comprises:
forming the electrode layer on the sensor substrate by means of a vapor deposition or sputtering process, so that the electrode layer covers a part of the hollow microneedle array;
forming the Prussian blue layer on a side of the electrode layer far away from the sensor substrate;
forming the enzyme layer on a side of the Prussian blue layer far away from the electrode layer; and
forming the biocompatible polymer layer on a side of the enzyme layer far away from the Prussian blue layer.

13. The using method according to claim 9, wherein
a partition area is provided between the working electrode and the counter electrode.

14. The using method according to claim 9, wherein before forming the working electrode and the counter electrode on the sensor substrate, the method further comprises:
forming a water-proof layer on the sensor substrate.

15. The using method according to claim 9, wherein in a process of penetrating through the tip of each microneedle in the hollow microneedle array, the method comprises:
penetrating through the tip of each microneedle in the hollow microneedle array by a metal needle array, wherein metal needles of the metal needle array are in a one-to-one correspondence with microneedles of the hollow microneedle array.

16. The using method according to claim 9, wherein
the mold is made of polydimethylsiloxane.
